(19) United States
(12) Patent Application Publication
(10) Pub. No.: US 2020/0000799 A1
(43) Pub. Date: Jan. 2, 2020

(54) METHOD AND USE OF BUPRENORPHINE IN TREATMENT OF CHRONIC PAIN

(71) Applicant: INTEGRATED MEDICAL SOLUTIONS, INC., Grand Rapids, MI (US)

(72) Inventor: Keith Javery, Grand Rapids, MI (US)

(21) Appl. No.: 16/436,651

(22) Filed: Jun. 10, 2019

Related U.S. Application Data
(60) Provisional application No. 62/682,377, filed on Jun. 8, 2018.

(51) Int. Cl. A61K 31/485; A61K 9/00; A61P 29/00

(52) U.S. Cl. CPC A61K 31/485; A61P 29/00; A61K 9/0019; A61K 9/0053

(57) ABSTRACT

A method of treating chronic pain of an individual is disclosed. The method can include determining an initial level of morphine milligram equivalents (MME) in the individual. The method can include determining a therapeutically effective dose of buprenorphine based on the initial level of MME. The method can include administering the therapeutically effective dose of buprenorphine to the individual.

### CROSS-REFERENCE TO RELATED APPLICATION(S)

This application claims priority to and the benefit of U.S. Provisional Application Patent Ser. No. 62/682,377, filed Jun. 8, 2018, the entire disclosure of which is hereby incorporated by reference.

### BACKGROUND

It has been well documented that pain is the most common reason a person visits a doctor's office. A mainstay of chronic pain management traditionally has involved the use of full mu-receptor agonist opioid medications. However, the opioid epidemic in the United States continues to be associated with the misuse and abuse of prescription opioid medications. Therefore, physicians find themselves in a conundrum about how to safely and effectively manage the disease of chronic pain.

Chronic pain affects hundreds of millions of people and is inherently difficult to manage. The Centers for Disease Control and Prevention (CDC) released guidelines that emphasize the use of non-opioid techniques to manage chronic pain and recommend limiting daily morphine milligram equivalents to certain levels in an effort to reduce the morbidity and mortality associated with opioids.

The U.S. Food and Drug Administration (FDA) has approved buprenorphine for the management of chronic pain. Buprenorphine is a partial mu agonist/kappa antagonist. Buprenorphine is commonly used in addiction medicine; however, buprenorphine has been shown to be a viable option for the management of chronic pain despite. Studies utilizing animal models have shown that buprenorphine has utility in treating pain but there is only a paucity of evidence to show the effectiveness of buprenorphine in the chronic pain population.

The method of transitioning patients from full mu agonist medications to sublingual buprenorphine has been plagued by issues with side effects, non-compliance with full mu agonist cessation, and high requirement of clinical/office staff effort to achieve a successful conversion to buprenorphine. Thus, there is a need for additional methods for transitioning patients and managing chronic pain.

### SUMMARY

This section provides a general summary of the present disclosure and is not a comprehensive disclosure of its full scope or all of its features, aspects, and objectives.

Disclosed herein are implementations of a method of treating chronic pain. The method can include administering a therapeutically effective dose of buprenorphine to an individual.

Also disclosed herein are implementations of a method of treating chronic pain of an individual. The method can include determining an initial level of morphine milligram equivalents (MME) in the individual. The method can include determining a therapeutically effective dose of buprenorphine based on the initial level of MME. The method can include administering the therapeutically effective dose of buprenorphine to the individual.

Also disclosed herein is a method of treating chronic pain of an individual. The method can include determining an initial level of morphine milligram equivalents (MME) in an individual, wherein the initial level of MME is between about 0-300 mg. The method can include determining a therapeutically effective dose of buprenorphine based on the initial level of MME, wherein the therapeutically effective dose of buprenorphine is between about 7.5 mg to about 10.75 mg per day. The method can include administering the therapeutically effective dose of buprenorphine to the individual.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a schematic drawing of buprenorphine in accordance with aspects of the present disclosure.

FIGS. 2-3 are flow charts of an exemplary patient study in accordance with aspects of the present disclosure.

FIGS. 4A-4I are line graphs illustrating results of an exemplary patient study in accordance with aspects of the present disclosure.

FIGS. 5-9 are bar graphs illustrating results of an exemplary study treating chronic pain and conversion in accordance with aspects of the present disclosure.

FIG. 10 is a flow chart of an exemplary method of treating of chronic pain of an individual in accordance with aspects of the present disclosure.

### DETAILED DESCRIPTION

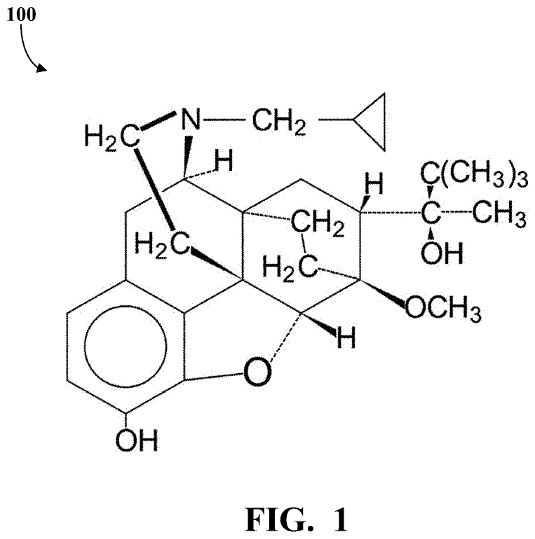

The present disclosure uses buprenorphine for the management of chronic pain. FIG. 1 illustrates the chemical composition of buprenorphine 100. Buprenorphine 100 can be used in parenteral and sublingual analgesic procedures. SL buprenorphine can be used for addiction maintenance therapy. TD buprenorphine can be used for moderate to severe pain. Buprenorphine 100 is a derivative of a morphine alkaloid thebaine. Buprenorphine 100 results in unique and complex pharmacology. Buprenorphine 100 can be used as an opioid partial agonists. Buprenorphine 100 can be used as a mixed agonist-antagonist.

Various non-selective, mixed agonist-antagonist opioid receptor modulators can be used to treat chronic pain. Mu can be a weak partial agonist, which binds with high affinity. Mu can be responsible for analgesia. Kappa can be a very week partial agonist/functional antagonist. Animal models may show anti-depressive, anxiolytic, and anti-addictive properties. Kappa can prevent psychotomimetic effects such as euphoria or dysphoria. Delta can be an antagonist with possible attenuation of a drug reward. Nociceptin/ORL-1 can be a weak partial agonist resulting in a lack of respiratory depression/ceiling effect.

Various combinations of buprenorphrine 100 can be used to treat chronic pain, drug dependence, or other medical issues. For example, a combination of buprenophrine 100 and naloxone can be used for the treatment of dependence. Various formulas are approved by the FDA for chronic pain. For example, the FDA has approved the following doses for an adult person for the treatment of pain: 2 mg bup=60 mg morphine, 5 mcg butrans=9 mg morphine, and 300 mcg belbuca=9 morphine.

The movement of drugs within the body can be analyzed to determine the effects on drugs in a person's body. For example, non-selective, mixed agonist-antagonist opioids can be metabolized by the person's liver (e.g., CYP3A4, norbuprenorphine). Glucoronidation by UGT1A1 and UGT2B7 can be excreted into bile. The elimination half-life can be between 20 and 73 hours, with a mean of 37 hours. Thus, there may be no little to no risk of accumulation causing renal impairment.

Buprenorphine 100 can be an effective drug for a variety of medical treatments. For example, buprenorphine 100 can be used to treat both cancer and non-cancer patients who have pain. Buprenorphine 100 can be as effective as full agonists morphine, hydromorphone, oxycodone, fentanyl, and methadone without some of the side effects cause by other drugs. For example, fentanyl can cause greater nausea and leads to greater discontinuation of use due to adverse events. Morphine can cause more constipation, nausea, and vomiting in patients. Buprenorphine 100 can have no difference in analgesia as fentanyl. Buprenorphine 100 can cause patients to have less analgesia than morphine.

Buprenorphine 100 can be fully effective in producing anti-nociception for neuropathic pain in animals. For example, Buprenorphine 100 can alleviate neuropathic pain-like behaviors in rats after spinal cord and peripheral nerve injury.

Buprenorphine 100 can block secondary hyperalgesia from central sensitization in humans. Buprenorphine 100 can also reduce post-thoracotomy pain. Dose-response relationship of opioids can be used in nociceptive and neuropathic postoperative pain. In one study, IV buprenorphine 100 was effective in pain reduction with response rate of up to 69% with doses from 35 to 70 mcg/hr.

Buprenorphine 100 can be used to treat a broader array of pain phenotypes. There may be less analgesic tolerance due to action on kappa opioid receptor antagonisms. Buprenorphine 100 can be used with other full mu agonists. Buprenorphine 100 can be combined with porphine and tramadol without loss of analgesia. Buprenorphine 100 can be supra-additive when combined with oxycodone and hydromorphone. Buprenorphine 100 can have a significant receptor reserve for other mu opioids. Buprenorphine 100 should not be combined with benzodiazepines. However, such a combination may be safer than a combination of buprenorphine 100 and methadone.

In animal models, buprenorphine 100 can reduce pain from a variety of mechanisms: formaline injection, cold temp tail flick, etc. In humans, buprenorphine 100 can be more effective over using fentanyl. For example buprenorphine 100 can be effective in treating attenuated bone pain, heat pain, pain related to nerve growth-factor injections, and cold pressor pain, whereas fentanyl may only be effective in treating cold pressor pain.

Buprenorphine 100 can lead to a smaller percentage of constipation. For example, drug-related constipation may be caused by buprenorphine 100 in 1-5% of patients, whereas full agonists may cause constipation in 40-80% of patients.

Buprenorphine 100 may not cause opioid-induced colic. Buprenorphine 100 can be used to treat chronic abdominal pain and chronic pancreatic.

Some drugs can create a ceiling effect on respiratory depression, but not on analgesia. For example, in animal models, buprenorphine 100 can have a significantly less effect on PaO2 and PaCO2 compared to methadone, morphine, and fentanyl. Buprenorphine 100 may not reduce time in expiration whereas full agonists do. In human studies, the safety index of buprenorphine 100 compared to fentanyl for the opioid-induced respiratory depression can be 14 versus 1.2. Fentanyl can have a linear dose-related analgesia and respiratory depression without a ceiling effect on either outcome. Buprenorphine 100 can have a linear analgesic effect and improved cutaneous pain 3-fold when doses were increased from 3 mcg/kg to 6 mcg/kg, but without causing any additional effect on respiration.

Buprenorphine 100 may have additional benefits. Buprenorphine 100 may result in less cognitive dysfunction. Buprenorphine 100 may not be immunosuppressive. Buprenorphine 100 may not suppress hypothalamic-pituitary-adrenal (HPA) or cause hypogonadism. Buprenorphine 100 may not significantly prolong QTc (i.e., the duration of a QT interval adjusted for the patient's heart rate. Prolonged QTc's can be associated with an increased risk of ventricular dysrhythmia and sudden death. Buprenorphine 100 can be less sudden than with methadone. Buprenorphine 100 may be a safe opioid to use in renal impairment.

Buprenorphine 100 can cause a decrease in a pain threshold from baseline after single or repeated administration of opioids. 90% of patients who present to pain centers are already taking opioids. Such patients may be clinically characterized by an increase in pain intensity over time, spreading of pain to other locations beyond initial painful site, or increase in pain sensation to external stimuli in setting of chronic opioid use. Many patients may have centralized pain disorders at risk for worsening hyperalgesia with opioids. Buprenorphine 100 may improve hyperalgesia.

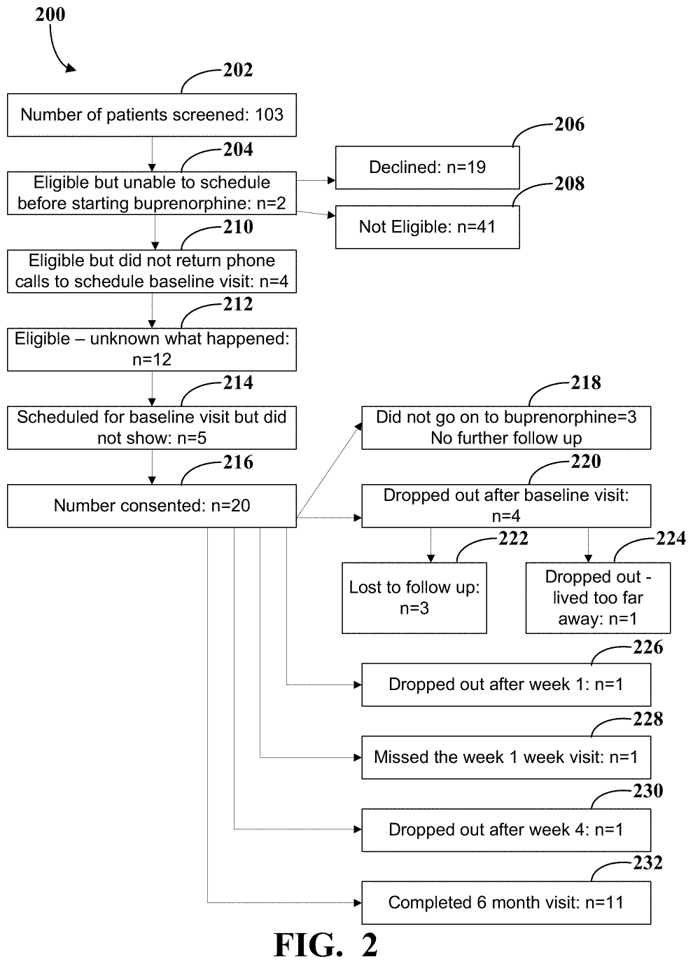

FIG. 2 illustrates a flow chart of a patient study 200. The patient study 200 illustrates patients screened, consented, and completed for a study. The patient study 200 generally illustrates assessed changes in phenotype and pressure sensitivity in patients with suspected opioid-induced hyperalgesia (OIH) (i.e., pain tolerance). The patient study 200 included measures of pain phenotyping and Quantitative Skin Testing (QST). Follow-up was performed at time increments or periods of 1, 4, and 8 weeks, and six months.

Steps 202 to 232 illustrate details of the patient study 200. One hundred and three (103) patients were screened. Two patients were eligible but unable to schedule before staring opioids. Nineteen patients declined using opioids. Forty-one patients were not eligible. Four patients were eligible but did not return phone calls to schedule a baseline visit. It is unknown what happened to twelve patients. Five patients were scheduled to set up a baseline visit but did not show. Twenty patients consented. Of those consenting patients, three patients did not go on to using opioids and 4 patients dropped out of the study after the baseline visit, of which three patients did not follow-up and one patient lived too far away and therefore dropped out of the patient study 200. Additionally, one patient dropped out after the first week, one patient missed the week one visit, one patient dropped out after week four, and eleven patients completed their 6-month visit. In other words, of the 103 patients screened, 20 patients consented to the study and 11 patients completed the entire patient study 200.

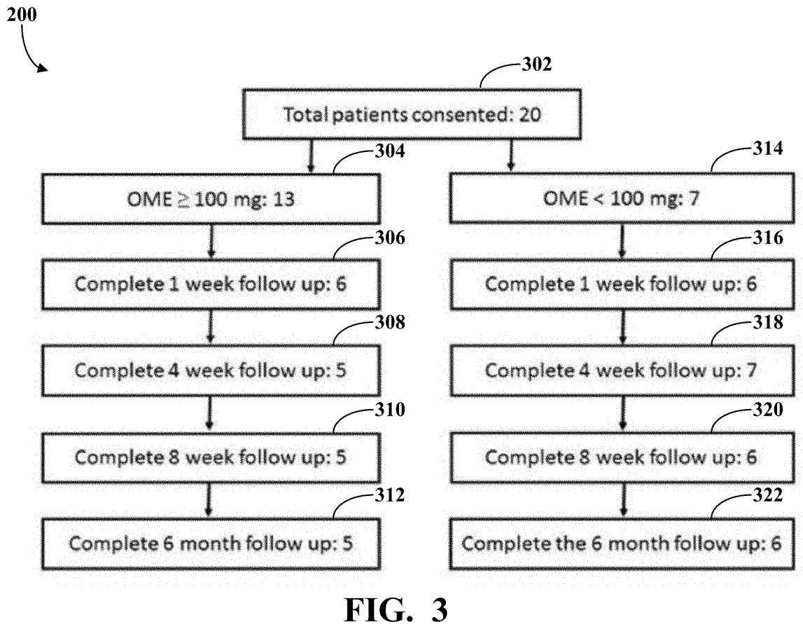

FIG. 3 illustrates the treatment of the 20 patients consenting to the patient study 200. As shown in steps 302 and 314, thirteen patients were treated with an opioid, such as an oral morphine equivalent (OME), dose of 100 mg or more and seven patients were treated with an OME dose of less than 100 mg. Steps 306 to 312 show that of the patients treated with the OME dose of 100 mg or more, six patients completed the week 1 follow-up appointment, five patients completed the week 4 follow-up appointment, five patients completed the week 8 follow-up appointment, and five patients completed the sixth month follow-up appointment. Steps 316 to 322 show that of the patients treated with the OME dose of less than 100 mg, six patients completed the week 1 follow-up appointment, seven patients completed the week 4 follow-up appointment, six patients completed the week 8 follow-up appointment, and six patients completed the sixth month follow-up appointment.

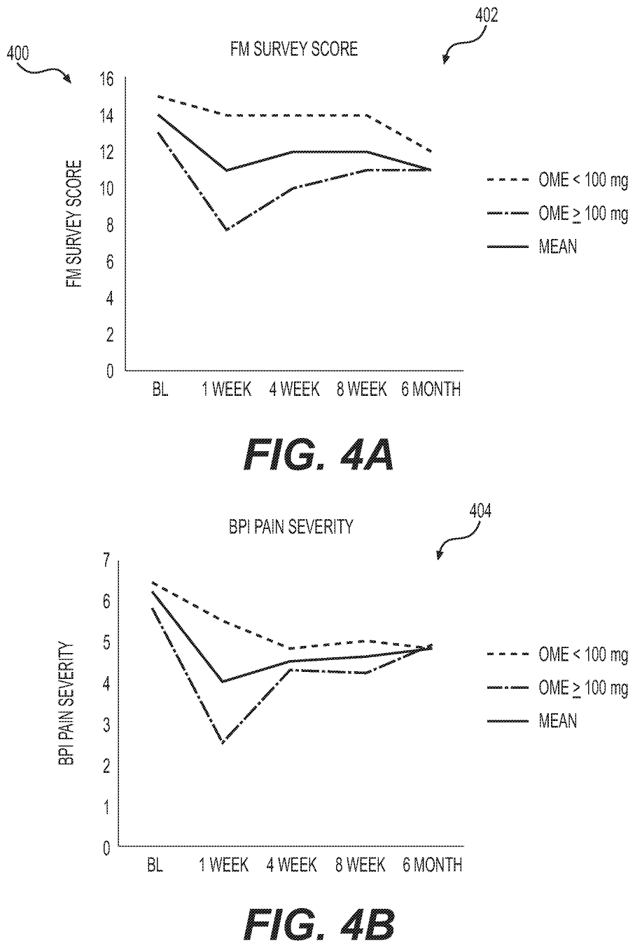

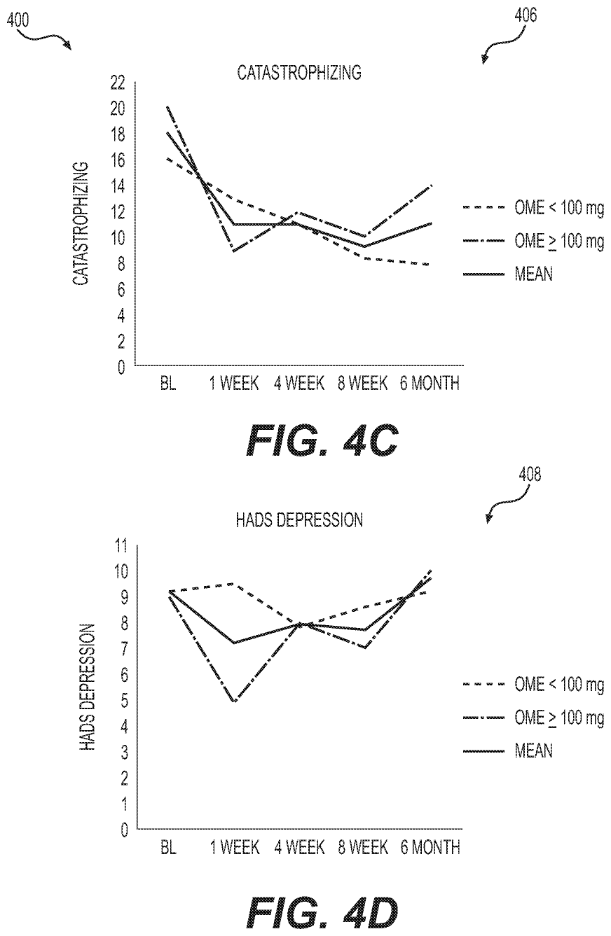

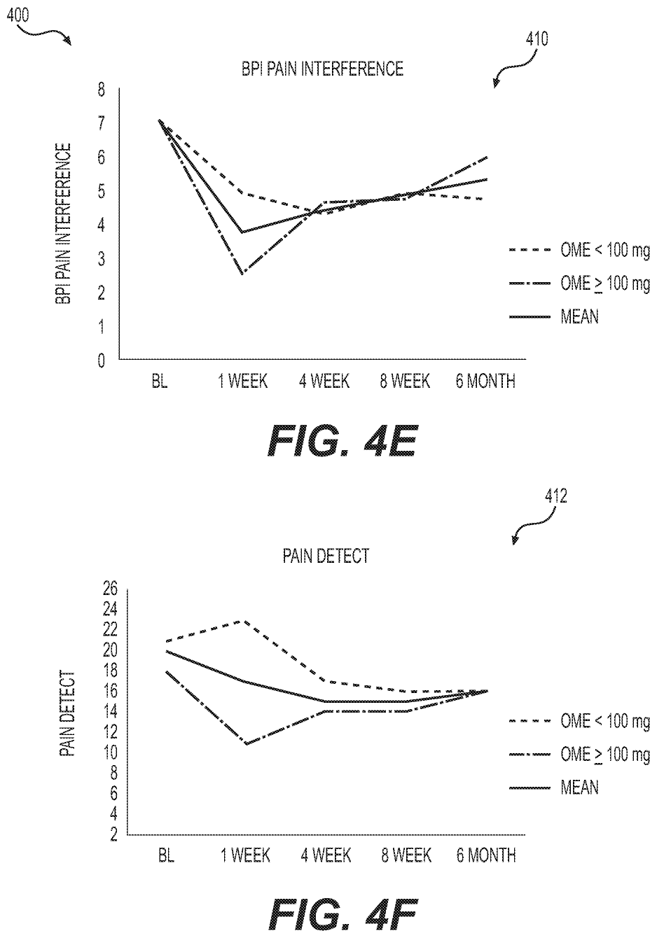

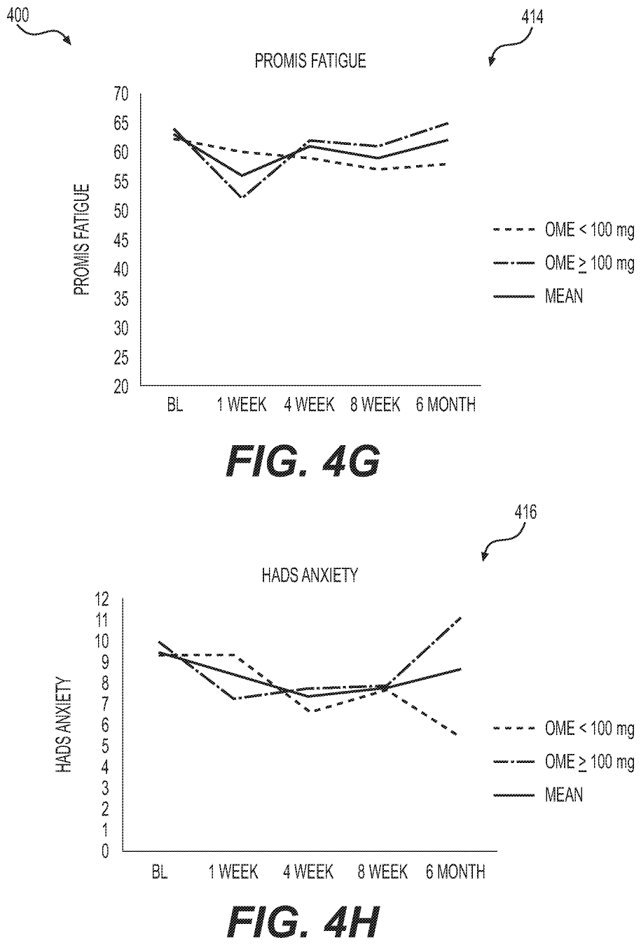

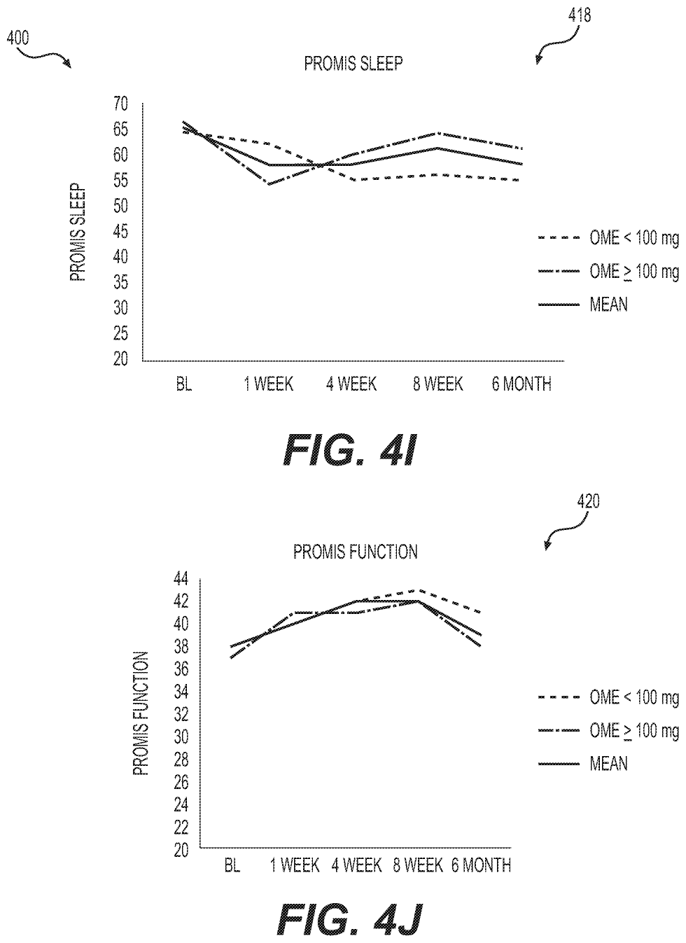

FIGS. 4A-4J show line graphs illustrating results 400 of the patient study 200. Line graphs 402-420 illustrate average values for four of the ten outcome variables from a baseline to the sixth-month follow-up for the entire patient group and for the patients treated with either 100 mg or more OME or less than 100 mg. Asterisks (*) can denote statistical significance of at least p greater than 0.05 for change in outcome from the baseline to the week 1 follow-up. For patients taking 100 mg or more of OME, there may have been statistically significant decreases in the fibromyalgia (FM) survey score, catastrophizing, pain severity, and depression (all ps less than 0.05) from baseline to the week 1 follow-up. Overall, higher opioid doses showed significant improvement for some measures including decreased pain severity and FM scores, fewer neuropathic features, less catastrophizing, fewer depressive symptoms, and improved function after one week with return to baseline after six months.

More specifically, line graph 402 in FIG. 4A shows a FM survey score of patients on OME<100 mg resulting in overall higher FM survey scores than patients on OME≥100 mg as analyzed over a six-month period. Line graph 404 in FIG. 4B shows Brief Pain Inventory (BPI) pain severity of patients on OME<100 mg resulting in overall higher BPI pain sensitivity than patients on OME≥100 mg as analyzed over a six-month period. Line graph 406 in FIG. 4C shows catastrophizing of patients on OME<100 mg resulting in either higher and lower catastrophizing scores over time than patients on OME≥100 mg as analyzed over a six-month period. Line graph 408 in FIG. 4D shows Hospital Anxiety and Depression Scale (HADS) depression of patients on OME<100 mg resulting in generally higher HADS (except for at the six-month mark) than patients on OME≥100 mg as analyzed over a six-month period. Line graph 410 in FIG. 4E shows BPI pain interference of patients on OME<100 mg resulting in generally higher BPI pain interference before week four, about the same between weeks four and eight and less after week eight than patients on OME≥100 mg as analyzed over a six-month period. Line graph 412 in FIG. 4F shows pain detected of patients on OME<100 mg resulting in overall higher pain detection than patients on OME≥100 mg as analyzed over a six-month period. At six months, the pain detected of patients in both groups was similar. Line graph 414 in FIG. 4G shows Patient-Reported Outcomes Measurement Information System (PROMIS) fatigue of patients on OME<100 mg resulting in generally higher PROMIS fatigue before week four and less after about week four than patients on OME≥100 mg as analyzed over a six-month period. Line graph 416 in FIG. 4H shows HADS anxiety of patients on OME<100 mg resulting in generally higher HADS anxiety before week four and less after about week four than patients on OME≥100 mg as analyzed over a six-month period. Line graph 418 in FIG. 41 shows hours of PROMIS sleep of patients on OME<100 mg resulting in generally higher number of hours of PROMIS sleep before week four and less after about week four than patients on OME≥100 mg as analyzed over a six-month period. Line graph 420 in FIG. 4J shows the level of PROMIS function of patients on OME<100 mg resulting in similar levels of PROMIS function before week four and greater levels after about week four than patients on OME≥100 mg as analyzed over a six-month period. The overall trend showed improved QST with higher opioid doses.

Buprenorphine 100 may be a potential replacement/substitute for treating chronic pain. The present disclosure uses methods for the conversion of patients on full mu agonist therapy to the use of buprenorphine 100. This innovative conversion technique can result in a high success rate. Patients can successfully convert on a mean dose of buprenorphine 100. A numerical pain level of a patient before and after conversion to buprenorphine 100 can be improved.

Furthermore, there is a correlation between pre-induction morphine milligram equivalents (MME) and overall reduction in Visual Analog Scale (VAS) scores post-induction.

An exemplary study of an observational prospective chart analysis of the conversion technique was conducted at the Javery Pain Institute. The Javery Pain Institute is a single-specialty private practice pain management center. An electronic medical record system was used for data extraction. Patients who were suffering from poorly managed chronic pain, who required or requested increasing opioid dosages, who demonstrated side effects from traditional mu agonists, or who were possibly suffering from opioid-induced hyperalgesia were deemed appropriate candidates for conversion. All patient provided informed consent after reviewing the risk, benefits, alternative therapies, possible side effects of buprenorphine 100 therapy, as well as the specific protocol for the novel accelerated buprenorphine induction procedure.

In this exemplary study, of the 229 patients' charts reviewed, 141 of the patients were females (62%) and 88 were males (38%). The patients were ages 26-96 with a mean age of 55.2. The mean daily morphine milligram equivalents (MME) was 85.4 mg, with a range of 0-1200 mg.

In this exemplary study, patients were instructed to discontinue their full mu agonist therapy for 48-72 hours prior to their induction procedure, which served as a washout period. During this time, patients were provided supportive medical care in the form of oral clonidine, vistaril, and/or clonezapam to ameliorate withdrawal symptoms. On the first day of a patient's induction at the clinic, the patient's pain scores (0-10) and Clinical Opiate Withdrawal Scale (COWS, 0-48) scores were recorded. An intravenous line was established. Blood pressure was monitored. Pulse oximetry was monitored. 30 micrograms of intravenous (IV) buprenorphine 100 was then administered via IV push at 10-minute intervals until there was a 50% reduction in both the patient's pain score and COWS score AND/OR a maximum total dose of 450 micrograms being administered.

Oral sublingual buprenorphine dosages were then calculated based on total IV buprenorphine 100 administered as follows:

All data were extracted from the patient electronic medical records in a rigorous manner. Data used included patient's age, sex, pre-induction morphine milligram equivalent (MME) based on most recent opioid prescription, COWS score during induction, total IV buprenorphine 100 doses, total SL buprenorphine dose, and medication side effects. Pain scores were recorded prior to conversion, at pre-induction, immediately post-induction, at 1-week follow-up, as well as their most recent follow-up in their standard clinic follow-up appointments.

This exemplary study assessed pain levels before and after conversion to buprenorphine 100. This exemplary study assessed the percentage of patients who successfully converted from full agonist opioids to buprenorphine 100. This exemplary study assessed the average buprenorphine 100 dose among those successfully converted.

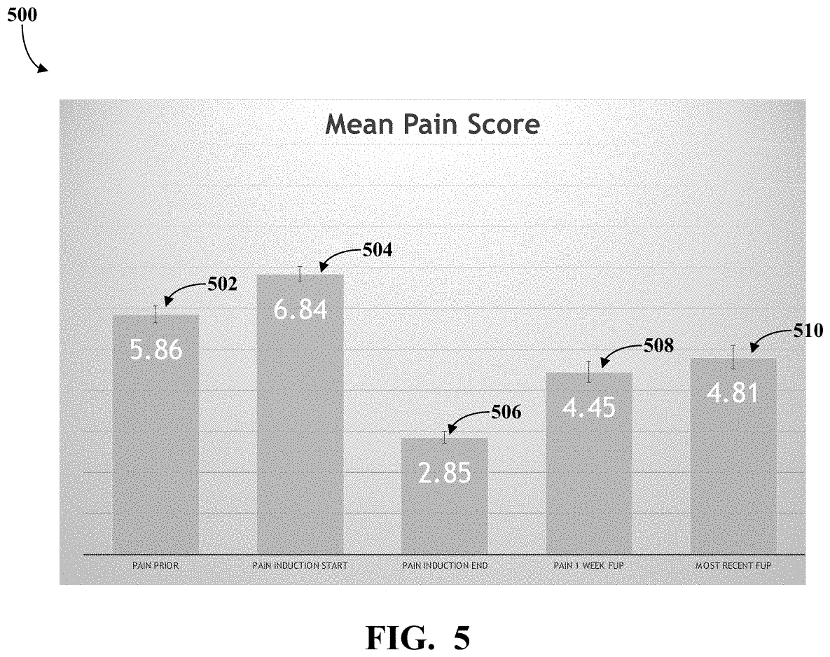

FIGS. 5-9 show bar graphs 500, 600, 700, 800, 900 illustrating results of the exemplary study treating chronic pain and conversion. Bar graph 500 in FIG. 5 includes mean pain score results. Specifically, bar 502 has a score of 5.86 for a pain prior, bar 504 has a score of 6.84 at a pain induction start, bar 506 has a 2.85 score at a pain induction end, bar 508 has a score of 4.45 at a week 1 follow-up appointment, and bar 510 has a score of 4.81 at a most recent follow-up appointment. Bar graph 500 shows a significant decrease in numerical pain scores that were sustained at six months.

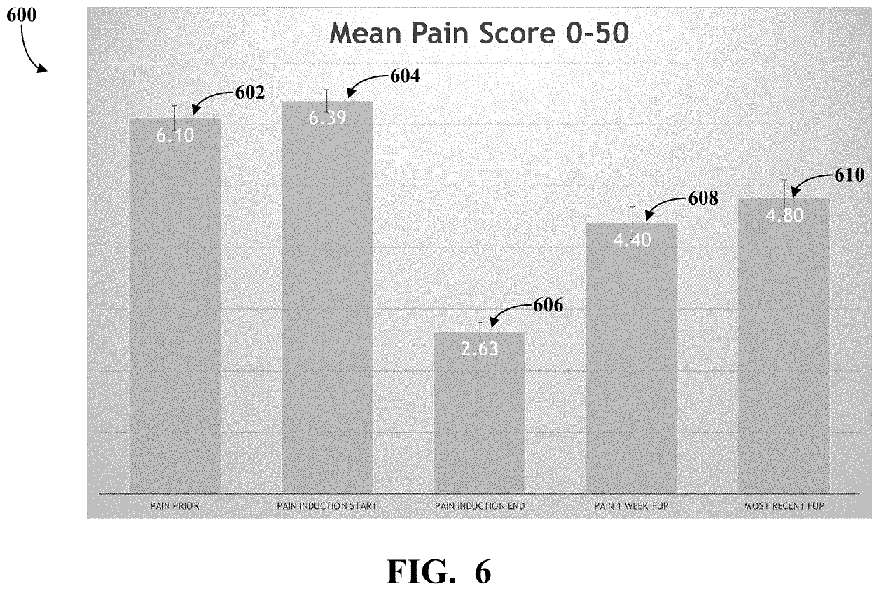

Bar graph 600 in FIG. 6 includes mean pain score results for patients with an initial amount of 0-50 mg of MME detected in their bodies. Specifically, bar 602 has a score of 6.10 for a pain prior, bar 604 has a score of 6.39 at a pain induction start, bar 606 has a 2.63 score at a pain induction end, bar 608 has a score of 4.40 at a week 1 follow-up appointment, and bar 610 has a score of 4.80 at a most recent follow-up appointment. Bar graph 600 shows a significant decrease in numerical pain scores for 0-50 mg that were sustained at six months.

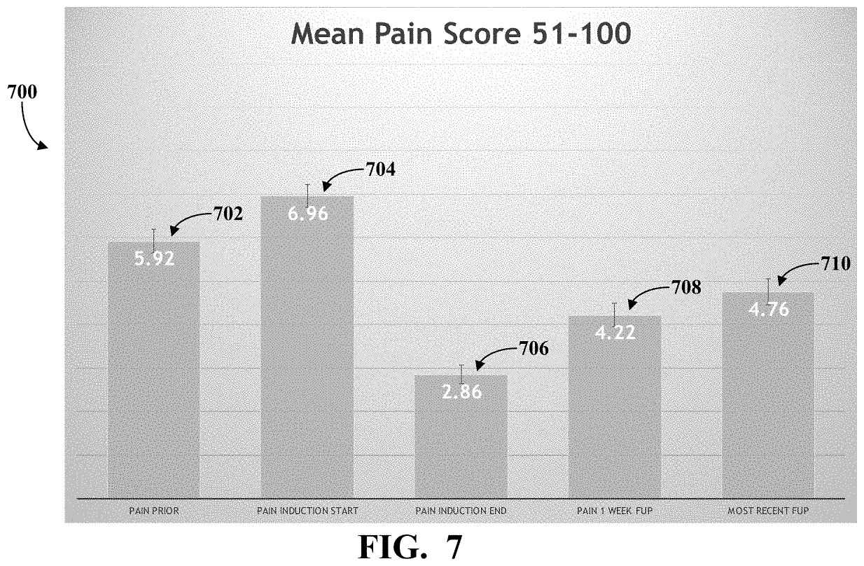

Bar graph 700 in FIG. 7 includes mean pain score results for patients with an initial amount of 51-100 mg of MME detected in their bodies. Specifically, bar 702 has a score of 5.92 for a pain prior, bar 704 has a score of 6.96 at a pain induction start, bar 707 has a 2.86 score at a pain induction end, bar 708 has a score of 4.22 at a week 1 follow-up appointment, and bar 710 has a score of 4.76 at a most recent follow-up appointment. Bar graph 700 shows a significant decrease in numerical pain scores for 51-100 mg that were sustained at six months. The response, however, is less dramatic than for 50-100 mg.

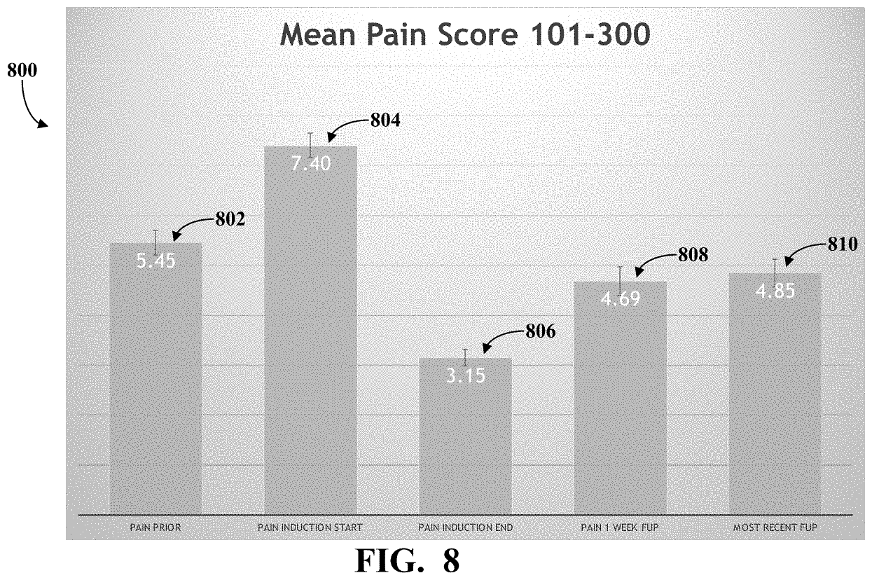

Bar graph 800 in FIG. 8 includes mean pain score results for patients with an initial amount of 101-300 mg of MME detected in their bodies. Specifically, bar 802 has a score of 5.45 for a pain prior, bar 804 has a score of 7.40 at a pain induction start, bar 808 has a 3.15 score at a pain induction end, bar 808 has a score of 4.69 at a week 1 follow-up appointment, and bar 810 has a score of 4.85 at a most recent follow-up appointment. Bar graph 800 shows a less robust response from patients with high initial MME. Patients with high initial MME may have been reporting uncontrolled pain or significant side effects with full mu agonists. Thus, the response may not have been as dramatic as patients having no or lower initial MME.

The results in FIGS. 5-8 show an 82% successful conversion rate. Seventeen patients were unsuccessful in the conversion. Of these seventeen patients, two had new acute injuries, four were unsuccessful due to other reasons, such as insurance restrictions or cost, and eleven failed due to side effects. The side effects may have included any of nausea, vomiting, headaches, lethargy, and nightmares.

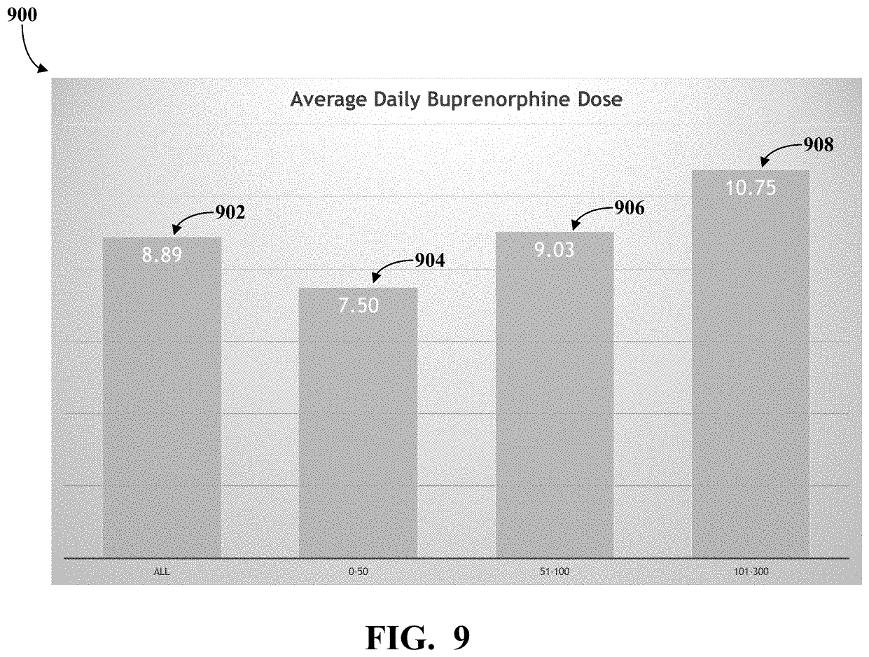

Bar graph 900 in FIG. 9 includes an average daily dose of buprenorphine 100. Specifically, bar 902 represents all patients and includes an average dose of 8.89 mg of buprenorphine 100. Bar 904 represents patients with an initial amount of MME between 0 and 50 mg and includes an average dose of 7.50 mg of buprenorphine 100. Bar 906 represents patients with an initial amount of MME between 51 and 100 mg and includes an average dose of 9.03 mg of buprenorphine 100. Bar 908 represents patients with an initial amount of MME between 101 and 300 mg and includes an average dose of 10.75 mg of buprenorphine 100. Thus, the response can be dose dependent when controlling for initial levels of MME.

Buprenorphine 100 can be an effective tool for chronic pain management. buprenorphine 100 can be used to treat nociceptive and neuropathic pain. Buprenorphine 100 has numerous benefits over full agonists. Buprenorphine 100 may improve OIH. Buprenorphine 100 can be safer and provide improved pain control with less side effects than using other drugs in chronic pain patients. For example, patients using buprenorphine 100 may have decreased renal impairment and decreased constipation. Buprenorphine 100 may also provide a ceiling effect for respiratory depression.

FIG. 1000 illustrates a flow chart 1000 of an exemplary method of treating chronic pain of an individual. More specifically, the flow chart 1000 shows the method of treating chronic pain comprising the steps of administering a therapeutically effective dose of buprenorphine 100 to an individual.

The therapeutically effective dose of buprenorphine 100 can vary amongst individuals, or patients. For example, the buprenorphine 100 administered to the individual can be in an amount of about 7.5 mg to about 10.75 mg per day, or any other desired amount. This range may be based on an average daily dosage of buprenorphine 100. The therapeutically effective dose may be more or less per day. The buprenorphine 100 administered to the individual can be in an amount of about 8.89 mg per day. The buprenorphine 100 administered to the individual can be in an amount of about 9.03 mg per day. The buprenorphine 100 administered to the individual can be in an amount of about 10.75 mg per day.

At step 1002, the initial level of MME can be determined in the individual. The initial level of MME may be between about 0-300 mg or any other detectable range. For example, the initial level of MME may be between about 0-50 mg. The initial level of MME may be between about 51-100 mg. The initial level of MME may be between about 101-300 mg.

At step 1004, the dose of buprenorphine 100 is determined. The therapeutically effective dose of buprenorphine 100 may be determined based on the level of MME in the individual. For example, if the initial level of MME detected in an individual is between about 0-300 mg, the therapeutically effective dose of buprenorphine 100 may be an amount of about 8.89 mg per day. The therapeutically effective dose of buprenorphine 100 may be a daily average of about 8.89 mg per day or any other desired amount. The actual therapeutically effective dose of buprenorphine 100 for an individual may be more or less each day the buprenorphine 100 is administered. If the initial level of MME detected in an individual is between about 0-50 mg, the buprenorphine 100 may be administered to the individual in an amount of about 7.5 mg per day or any other desired amount. If the initial level of MME detected in an individual is between about 51-100 mg, the buprenorphine 100 may be administered to the individual in an amount of about 9.03 mg per day or any other desired amount. If the initial level of MME detected in an individual is between about 101-300 mg, the buprenorphine 100 may be administered to the individual in an amount of about 10.75 mg per day or any other desired amount.

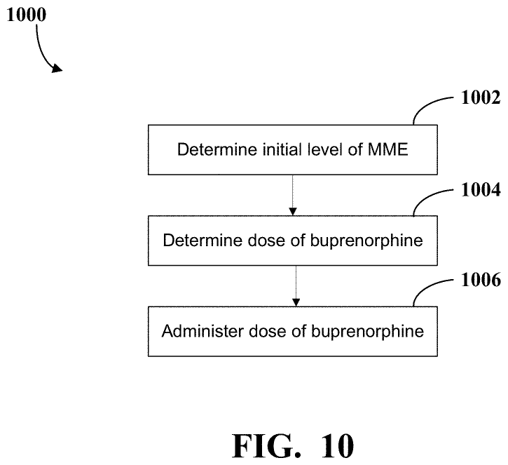

At step 1006, the dose of buprenorphine 100 is administered. The buprenorphine 100 may be administered to individuals in various forms using various procedures. The buprenorphine 100 can be administered at least one of orally, parenterally, topically, transdermally, or any other desired procedure. The oral administration may be a capsule, tablet, aerosol spray, or any other desired form. The buprenorphine 100 can be parentarally administered subcutaneously, intraveously, intramuscularly, or by any other desirable procedure. The methods described in FIG. 10 can include additional and/or fewer components and/or steps in an alternative order and are not limited to those illustrated in this disclosure.

In one exemplary method of treating chronic pain of an individual, the method can comprise the step of determining an initial level of MME in the individual. The initial level of MME detected can be between about 0-300 mg or any other detectable amount. The method can comprise the step of determining a therapeutically effective dose of buprenorphine 100 based on the initial level of MME. The therapeutically effective dose of buprenorphine 100 may be between about 7.5 mg to about 10.75 mg per day or any other desired amount. The method can comprise the step of administering the therapeutically effective dose of buprenorphine 100 to the individual. If the initial level of MME detected in an individual is between about 0-50 mg, an average of about 7.5 mg of the buprenorphine can be administered to the individual per day. If the initial level of MIME detected in an individual is between about 51-100 mg, an average of about 9.03 mg of the buprenorphine can be administered to the individual per day. If the initial level of MIME detected in the individual is between about 101-300 mg, an average of about 10.75 mg of the buprenorphine can be administered to the individual per day.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of treating chronic pain, the method comprising the steps of administering a therapeutically effective dose of buprenorphine to an individual.

2. The method of claim 1, wherein the buprenorphine is administered to the individual in an amount of about 7.5 mg to about 10.75 mg per day.

3. The method of claim 1, wherein the buprenorphine is administered to the individual in an amount of about 7.5 mg per day.

4. The method of claim 1, wherein the buprenorphine is administered to the individual in an amount of about 8.89 mg per day.

5. The method of claim 1, wherein the buprenorphine is administered to the individual in an amount of about 9.03 mg per day.

6. The method of claim 1, wherein the buprenorphine is administered to the individual in an amount of about 10.75 mg per day.

7. The method of claim 1, wherein the buprenorphine is administered at least one of orally, parenterally, topically, and transdermally.

8. The method of claim 7, wherein the oral administration is by a capsule, tablet, or aerosol spray.

9. The method of claim 7, wherein the buprenorphine is parentarally administered subcutaneously, intraveously, or intramuscularly.

10. The method of claim 1, further comprising:
determining an initial level of morphine milligram equivalents (MME) in the individual;
determining the therapeutically effective dose of buprenorphine based on the level of MME.

11. A method of treating chronic pain of an individual, the method comprising the steps of:
determining an initial level of morphine milligram equivalents (MME) in the individual;
determining a therapeutically effective dose of buprenorphine based on the level of MME; and
administering the therapeutically effective dose of buprenorphine to the individual.

12. The method of claim 11, wherein the initial level of MME is between about 0-300 mg.

13. The method of claim 12, wherein the buprenorphine is administered to the individual in an amount of about 8.89 mg per day.

14. The method of claim 11, wherein the buprenorphine is administered to the individual in an amount of about 7.5 mg to about 10.75 mg per day.

15. The method of claim 11, wherein the initial level of MME is between about 0-50 mg, and wherein the buprenorphine is administered to the individual in an amount of about 7.5 mg per day.

16. The method of claim 11, wherein the initial level of MME is between about 51-100 mg, and wherein the buprenorphine is administered to the individual in an amount of about 9.03 mg per day.

17. The method of claim 11, wherein the initial level of MME is between about 101-300 mg, and wherein the buprenorphine is administered to the individual in an amount of about 10.75 mg per day.

18. A method of treating chronic pain of an individual, the method comprising the steps of:
determining an initial level of morphine milligram equivalents (MME) in the individual, wherein the initial level of MME is between about 0-300 mg;
determining a therapeutically effective dose of buprenorphine based on the initial level of MME, wherein the therapeutically effective dose of buprenorphine is between about 7.5 mg to about 10.75 mg per day; and
administering the therapeutically effective dose of buprenorphine to the individual.

19. The method of claim 18, wherein if the initial level of MME is between about 0-50 mg, an average of about 7.5 mg of the buprenorphine is administered to the individual per day;
wherein if the initial level of MME is between about 51-100 mg, an average of about 9.03 mg of the buprenorphine is administered to the individual per day; and
wherein if the initial level of MME is between about 101-300 mg, an average of about 10.75 mg of the buprenorphine is administered to the individual per day.

20. The method of claim 18, wherein the buprenorphine is administered at least one of orally, parenterally, topically, and transdermally.
